(19) United States
(12) Patent Application Publication
(10) Pub. No.: US 2023/0285887 A1
(43) Pub. Date: Sep. 14, 2023

(54) NANOFIBER FILTER AND METHOD FOR MANUFACTURING SAME

(71) Applicant: SAMSUNG ELECTRONICS CO., LTD., Suwon-si (KR)

(72) Inventors: Soomin Park, Suwon-si (KR); Saemi Kim, Suwon-si (KR); Heejin Park, Suwon-si (KR); Sungwon Kim, Suwon-si (KR); Jeeyeon Kim, Suwon-si (KR); Changbae Lim, Suwon-si (KR); Yeonkyoung Jung, Suwon-si (KR); Yongwon Jeong, Suwon-si (KR)

(73) Assignee: SAMSUNG ELECTRONICS CO., LTD., Suwon-si (KR)

(21) Appl. No.: 18/199,077

(22) Filed: May 18, 2023

Related U.S. Application Data
(63) Continuation of application No. PCT/KR2021/014089, filed on Oct. 13, 2021.

(30) Foreign Application Priority Data
Nov. 18, 2020 (KR) 10-2020-0154430

(51) Int. Cl. B01D 46/54; B01D 39/16

(52) U.S. Cl. CPC B01D 46/546; B01D 39/1623; B01D 2275/10; B01D 2239/025; B01D 2239/10; B01D 2239/065; B01D 2239/0631

(57) ABSTRACT

A nanofiber filter includes: a first support having a front surface and a rear surface opposite to the front surface; a first nanofiber filter layer disposed on the rear surface of the first support; a second nanofiber filter layer disposed on a rear surface of the first nanofiber filter layer; a third nanofiber filter layer disposed on a rear surface of the second nanofiber filter layer; and a second support disposed on a rear surface of the third nanofiber filter layer.

### CROSS-REFERENCE TO RELATED APPLICATION(S)

This application is a bypass continuation application of International Application No. PCT/KR2021/014089, filed on Oct. 13, 2021, which is based on and claims priority to Korean Patent Application No. 10-2020-0154430, filed on Nov. 18, 2020, in the Korean intellectual property office, the disclosures of which are incorporated by reference herein in their entireties.

### BACKGROUND

### 1. Field

The disclosure relates to a functional nanofiber filter and a method for manufacturing the same.

### 2. Description of Related Art

As awareness of environmental issues, such as air and water contamination and water shortage, increases with the advancement of industry, the development of air purification devices and water treatment devices that may efficiently separate and remove contaminated water and air is required. These devices use a filter capable of separating impurities and contaminants and discharging filtered clean air and clean water.

For example, such filters include air purifier filters, mask filters, or photocatalytic filters.

The air purifier filters may be used to remove fine dust for air purification, but may not be suitable for sterilizing viruses and bacteria smaller than fine dust. Further, the mask filters use the principle of collecting particles with electrostatic force from static electricity. However, the mask filters have the shortcoming of losing the filtering capability if the electrostatic force is lost. Therefore, the mask filters are suitable for one-time use but inappropriate for reuse. The photocatalytic filters may sterilize organic substances, viruses, and bacteria by radiating long-wavelength ultraviolet rays (UV-A). The photocatalytic filters lack a function of collecting viruses for effective sterilization.

An air purifier or mask requires a filter to sterilize viruses, a filter to remove harmful gases, odors and bad breath, and a filter to remove moisture and vapor. For example, since the air purifier and the mask require individual filters to implement the functions described above, the manufacturing costs of the filters may increase, and thus the manufacturing costs of the products may increase.

Further, the mask filter is mostly discarded after use, and when reused, the filter function is degraded. Therefore, since the mask filters are discarded after use, the waste increases, which leads to a waste of resources and an increase in purchase cost and environmental contamination.

### SUMMARY

Provided are a nanofiber filter and a method for manufacturing the same that may implement virus removal, harmful gas removal, odor removal, bad breath removal, moisture removal and water vapor removal by one filter.

According to an aspect of the disclosure, a nanofiber filter includes: a first support having a front surface and a rear surface opposite to the front surface; a first nanofiber filter layer having a front surface and a rear surface, wherein the front surface of the first nanofiber filter layer is disposed on the rear surface of the first support; a second nanofiber filter layer having a front surface and a rear surface, wherein the front surface of the second nanofiber filter layer is disposed on a rear surface of the first nanofiber filter layer; a third nanofiber filter layer having a front surface and a rear surface, wherein the front surface of the third nanofiber filter layer is disposed on a rear surface of the second nanofiber filter layer; and a second support disposed on the rear surface of the third nanofiber filter layer.

Each the first support and the second support may include at least one of a transparent breathable support or a translucent breathable support.

Each of the first support and the second support may include at least one of a hydrophilic polymer material, a hydrophobic polymer material, or a decomposable polymer material.

Each of the first support and the second support may include at least one of a metal mesh member or a breathable transparent film.

The first nanofiber filter layer may include an electrospun photocatalytic nanomaterial mixture.

The photocatalytic nanomaterial mixture may include at least one of UV-sensitive photocatalytic nanoparticles or visible light-sensitive photocatalytic nanoparticles.

The second nanofiber filter layer may include an electrospon gas suction material mixture.

The third nanofiber filter layer may include an electrospun hygroscopic nanomaterial mixture.

The nanofiber filter may be configured to filter at least one of a virus, gas, odor, moisture, and water vapor from air.

According to an aspect of the disclosure, a method for manufacturing a nanofiber filter, includes: forming a first nanofiber filter layer on a rear surface of a first support by electrospinning a photocatalytic nanomaterial mixture; forming a second nanofiber filter layer on a rear surface of the first nanofiber filter layer by electrospinning a gas suction material mixture; forming a third nanofiber filter layer on a rear surface of the second nanofiber filter layer by electrospinning a hygroscopic nanomaterial mixture; and disposing a second support on a rear surface of the third nanofiber filter layer.

Each of the first support and the second support may include at least one of a transparent breathable support or a translucent breathable support.

Each of the first support and the second support may include at least one of a hydrophilic polymer material, a hydrophobic polymer material, or an easily decomposable polymer material.

Each of the first support and the second support may include at least one of a metal mesh member or a breathable transparent film.

The photocatalytic nanomaterial mixture may include at least one of UV-sensitive photocatalytic nanoparticles or visible light-sensitive photocatalytic nanoparticles.

The nanofiber filter may be configured to filter at least one of a virus, gas, odor, moisture, and water vapor from air.

According to one or more embodiments of the disclosure, the nanofiber filter may be configured as a single filter by sequentially disposing a first nanofiber filter layer, a second nanofiber filter layer, and a third nanofiber filter layer, thereby sequentially implementing sterilization of viruses contained in the air, harmful gas removal, odor removal, bad breath removal, moisture removal, and water vapor removal.

Further, since the nanofiber filter according to one or more embodiments of the disclosure does not require a plurality of filters which may be conventionally required to implement the plurality of functions described above, may be is possible to save product manufacturing costs and simplify the product assembly process.

Further still, the nanofiber filter may allow the used mask to be reused by washing using a regeneration device, saving the wearer costs for purchasing new masks and reducing environmental contamination due to waste of masks.

### BRIEF DESCRIPTION OF THE DRAWINGS

FIG. 1 is an exploded perspective view illustrating a configuration of a nanofiber filter according to various embodiments of the disclosure;

FIG. 2 is a side view illustrating a configuration of a nanofiber filter according to various embodiments of the disclosure;

FIG. 3 is a flowchart illustrating a method for manufacturing a nanofiber filter according to various embodiments of the disclosure;

FIG. 4 is a view illustrating a state in which a nanofiber filter is applied to a mask according to various embodiments of the disclosure;

FIG. 5 is a view illustrating a state in which a nanofiber filter is applied to an air purifier according to various embodiments of the disclosure;

FIG. 6 is a side view illustrating a configuration of a nanofiber filter according to other various embodiments of the disclosure;

FIG. 7 is a flowchart illustrating a method for manufacturing a nanofiber filter according to other various embodiments of the disclosure;

FIG. 8 is a view illustrating a regeneration device of a nanofiber filter according to various embodiments of the disclosure; and

FIG. 9 is a view illustrating another embodiment of a regeneration device of a nanofiber filter according to various embodiments of the disclosure.

### DETAILED DESCRIPTION

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order),It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

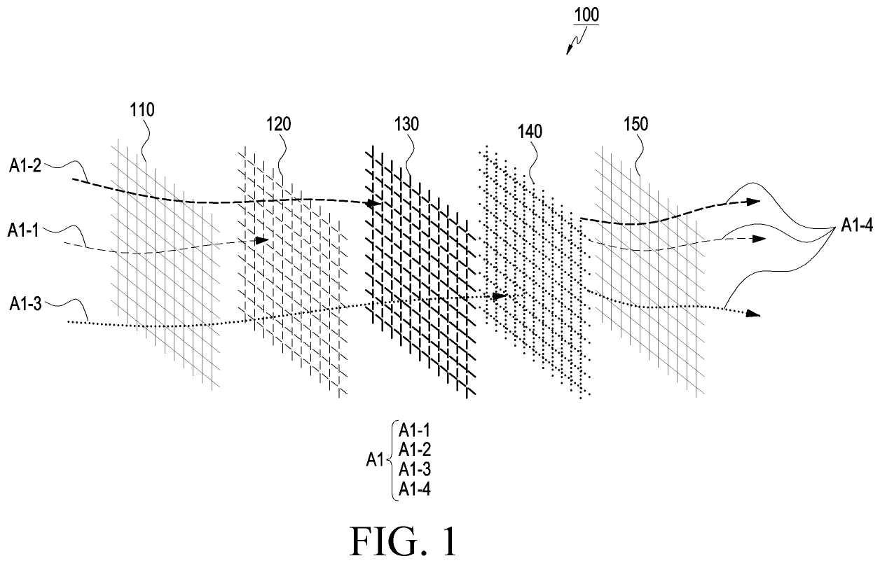

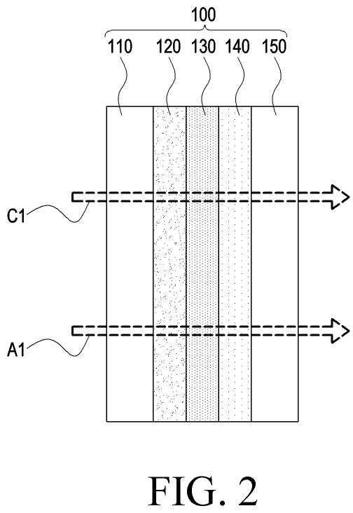

FIG. 1 is an exploded perspective view illustrating a nanofiber filter 100 according to various embodiments of the disclosure. FIG. 2 is a side view illustrating a nanofiber filter 100 according to various embodiments of the disclosure.

Referring to FIGS. 1 and 2, according to various embodiments, a nanofiber filter 100 may include first and second supports 110 and 150 and first, second, and third nanofiber filter layers 120, 130, and 140. The first and second supports 110 and 150 may include a front surface and a rear surface opposite to the front surface. The first, second, and third nanofiber filter layers 120, 130, and 140 may also include a front surface and a rear surface opposite to the front surface.

The front surface of the first nanofiber filter layer 120 may be disposed facing the rear surface of the first support 110. The front surface of the second nanofiber filter layer 130 may be disposed facing the rear surface of the first nanofiber filter layer 120. The front surface of the third nanofiber filter layer 140 may be disposed facing the rear surface of the second nanofiber filter layer 130. The front surface of the second support 150 may be disposed facing the rear surface of the third nanofiber filter layer 140. Therefore, in the nanofiber filter 100, the first nanofiber filter layer 120 may be sequentially disposed on the rear surface of the first support 110, the second nanofiber filter layer 130 may be sequentially disposed on the rear surface of the first nanofiber filter layer 120, the third nanofiber filter layer 140 may be sequentially disposed on the rear surface of the second nanofiber filter layer 130, and the second support 150 may be sequentially disposed on the rear surface of the third nanofiber filter layer 140.

For example, in the nanofiber filter 100, the first, second, and third nanofiber filter layers 120, 130, and 140 and the second support 150 may be sequentially disposed toward the rear surface of the first support 110.

According to various embodiments, the first support 110, the first, second, and third nanofiber filter layers 120, 130, and 140, and the second support 150 may be attached to each other through an adhesive member or be disposed without the adhesive member. The rear surface of the first support may be adhered to the front surface of the first nanofiber layer. The rear surface of the first nanofiber layer may be adhered to the front surface of the second nanofiber layer. The rear surface of the second nanofiber layer may be adhered to the front surface of the third nanofiber layer. The rear surface of the third nanofiber layer may be adhered to the front surface of the second support.

According to various embodiments, the first and second supports 110 and 150 may include at least one of transparent breathable supports or translucent breathable supports. For example, various breathable supports transmitting air A1 or light C1 may be applied as the first and second supports 110 and 150. In this embodiment, an example in which the first and second supports 110 and 150 are transparent breathable supports is described.

According to various embodiments, the first and second supports 110 and 150 may include at least one of a hydrophilic polymer material, a hydrophobic polymer material, or an easily decomposable polymer material. For example, the hydrophilic polymer material may include polyacrylonitrile (PAN). The hydrophilic polymer material may be other various hydrophilic polymer materials than polyacrylonitrile (PAN).

Further, the hydrophobic polymer material may include at least one of polyvinylylene fluoride (PVDF) or polyethylene terephthalate (PET). Likewise, the hydrophobic polymer material may be other various hydrophobic polymer materials than the above-disclosed material.

Further, the easily decomposable polymer material may include polyvinyl alcohol (PVA). Similarly, the easily decomposable polymer material may be other various readily decomposable polymer materials than polyvinyl alcohol (PVA).

According to various embodiments, the first and second supports 110 and 150 may include at least one of a metal mesh member or a breathable transparent film to transmit air A1 and light C1. In this embodiment, an example in which the first and second supports 110 and 150 are breathable transparent films is described.

According to various embodiments, the first support 110 may be disposed on the front surface of the nanofiber filter 100. The second support 150 may be disposed on the rear surface of the nanofiber filter 100. The first, second, and third nanofiber filter layers 120, 130, and 140 may be sequentially disposed between the rear surface of the first support 110 and the front surface of the second support 150.

In this state, the first support 110 may transmit air A1 including virus A1-1, harmful gas A1-2, odor A1-2, bad breath A1-2, moisture A1-3 and water vapor A1-3 and sequentially transfer it to the first, second, and third nanofiber filter layers 120, 130, and 140. As such, the air A1-4 passing through the first, second and third nanofiber filter layers 120, 130 and 140 may pass through the second support 150 and be discharged to the outside of the second support 150.

In this case, the first nanofiber filter layer 120 may be formed by electrospinning a photocatalytic nanomaterial mixture. For example, the air A1 passing through the first support 110 may be firstly filtered by the first nanofiber filter layer 120 while passing through the first nanofiber filter layer 120, sterilizing the virus A1-1 contained in the air. The air A1 becomes filtered upon passing through the first nanofiber filter layer 120.

The firstly purified air A1 may pass through the second nanofiber filter layer 130. The second nanofiber filter layer 130 may be formed by electrospinning a gas suction material mixture. For example, while the firstly purified air A1 passes through the second nanofiber filter layer 130, harmful gas A1-2, odor A1-2, and bad breath A1-2 contained in the air A1 may be simultaneously filtered secondly and removed by the second nanofiber filter layer 130. The air A1 becomes secondly filtered upon passing through the second nanofiber filter layer 130.

The firstly and secondly purified air A1 may pass through the third nanofiber filter layer 140. For example, the third nanofiber filter layer 140 may be formed by electrospinning a hygroscopic nanomaterial mixture. While the firstly and secondly purified air A1 passes through the third nanofiber filter layer 140, the moisture A1-3 and water vapor A1-3 contained in the air A1 may be simultaneously filtered thirdly and removed by the third nanofiber filter layer 140. The air A1 becomes thirdly filtered upon passing through the third nanofiber filter layer 140.

The firstly, secondly, and thirdly purified air A1-4 may pass through the second support 150 while being simultaneously transferred thereto, and may be discharged to the outside of the second support 150.

The air A1-4 passing through the second support 150 may be discharged to the outside, with the virus A1-1 sterilized, and the harmful gas A1-2, odor A1-2, bad breath A1-2, moisture A1-3, and water vapor A1-3 removed by the first, second, and third nanofiber filter layers 120, 130, and 140,

According to various embodiments, the first, second and third nanofiber filter layers 120, 130 and 140 may be manufactured by an electrospinning device described below. For example, the electrospinning device may include a solution supply unit, a spinning nozzle, and breathable supports 110 and 150.

For example, the solution supply unit may supply a photocatalytic nanomaterial mixture to the spinning nozzle to manufacture the first nanofiber filter layer 120. In this case, the spinning nozzle may form an electric field by applying a high voltage. For example, the spinning nozzle may deform hemispherical liquid droplets into cone shapes by the electric field and emit them. For example, the spinning nozzle may manufacture the first nanofiber filter layer 120 by emitting the photocatalytic nanomaterial mixture in the form of a thread having a diameter of tens to hundreds of nm by the electric field.

According to various embodiments, the photocatalytic nanomaterial mixture may include at least one of UV-sensitive photocatalytic nanoparticles or visible light-sensitive photocatalytic nanoparticles.

Further, the solution supply unit may supply a gas suction material mixture to the spinning nozzle to manufacture the second nanofiber filter layer 130. In this case, the spinning nozzle may form an electric field by applying a high voltage. For example, the spinning nozzle may manufacture the second nanofiber filter layer 130 by emitting the gas suction material mixture in the form of a thread having a diameter of tens to hundreds of nm by the electric field.

Further, the solution supply unit may supply a hygroscopic nanomaterial mixture to the spinning nozzle to manufacture the third nanofiber filter layer 140. In this case, the spinning nozzle may form an electric field by applying a high voltage. For example, the spinning nozzle may manufacture the third nanofiber filter layer 140 by emitting the gas suction material mixture in the form of a thread having a diameter of tens to hundreds of nm by the electric field.

As such, the nanofiber filter 100 is configured as a single filter by sequentially disposing the first, second and third nanofiber filter layers 120, 130 and 140, so that the nanofiber filter 100 may sequentially perform sterilization of the virus A1-1 contained in the air A1, removal of the harmful gas A1-2, removal of the odor A1-2, removal of the bad breath A1-2, removal of the moisture A1-2, and removal of the water vapor A1-3. Therefore, the nanofiber filter 100 may further enhance the function of purifying the air A1.

Further, since the single nanofiber filter 100 does not require a plurality of filters which are conventionally required to perform the plurality of functions described above, the manufacturing costs of the product may be reduced.

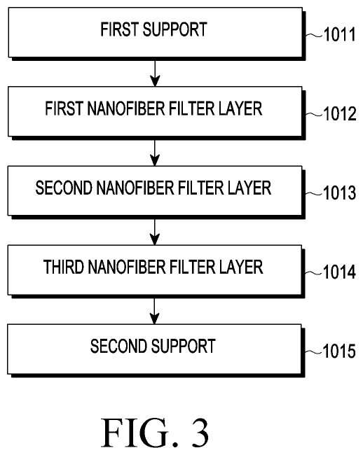

FIG. 3 is a flowchart illustrating a method for manufacturing a nanofiber filter 100 according to various embodiments of the disclosure.

Referring to FIG. 3, according to various embodiments, the first support 110 may be prepared to manufacture the nanofiber filter 100 (1011). For example, the first support 110 may include a front surface and a rear surface opposite to the front surface. The first support 110 may include at least one of a transparent breathable support and a translucent breathable support. In this embodiment, the first support 110 is described as a transparent breathable support.

According to various embodiments, the first support 110 may include at least one of a hydrophilic polymer material, a hydrophobic polymer material, or an easily decomposable polymer material. Further, the first support 110 may include at least one of a metal mesh member or a breathable transparent film. In this embodiment, an example in which the first support 110 is a breathable transparent film is described.

The first nanofiber filter layer 120 may be formed by electrospinning the photocatalytic nanomaterial mixture. The first nanofiber filter layer 120 may be sequentially disposed on the rear surface of the first support 110. For example, the rear surface of the first support 110 and the front surface of the first nanofiber filter layer 120 may be disposed to face each other (1012).

The second nanofiber filter layer 130 may be formed by electrospinning the gas suction material mixture. The second nanofiber layer 130 may be sequentially disposed on the rear surface opposite to the front surface of the first nanofiber filter layer 120. For example, the rear surface of the first nanofiber filter layer 120 and the front surface of the second nanofiber filter layer 130 may be disposed to face each other (1013).

The third nanofiber filter layer 140 may be formed by electrospinning the hygroscopic nanomaterial mixture. The third nanofiber filter layer may be sequentially disposed on the rear surface opposite to the front surface of the second nanofiber filter layer 130. For example, the rear surface of the second nanofiber filter layer 130 and the front surface of the third nanofiber filter layer 140 may be disposed to face each other (1014).

The second support 150 may be sequentially disposed on the rear surface opposite to the front surface of the third nanofiber filter layer 140. For example, the rear surface of the third nanofiber filter layer 140 and the front surface of the second support 150 may be disposed to face each other (1015).

According to various embodiments, the second support 150 may include a transparent breathable support.

For example, the first support 110 may be disposed on the front surface of the nanofiber filter 100, and the second support 150 may be disposed on the rear surface of the nanofiber filter 100. The first, second and third nanofiber filter layers 120, 130, and 140 may be sequentially disposed between the first and second supports 110 and 150.

In the nanofiber filter 100, when the air A1 is introduced through the first support 110, the first support 110 may transmit the air A1 while simultaneously transferring the air to the first nanofiber filter layer 120. The air A1 may be firstly filtered by the first nanofiber filter layer 120 while simultaneously passing through the first nanofiber filter layer 120, sterilizing the virus A1-1 contained in the air A1. The firstly purified air A1 may be transferred to the second nanofiber filter layer 130 while simultaneously passing through the second nanofiber filter layer 130. The sterilized air A1 may be secondarily filtered by the second nanofiber filter layer 130 while simultaneously passing through the second nanofiber filter layer 130, removing the harmful gas A1-2, odor A1-2, and bad breath A1-2 contained in the air A1. The so-purified firstly and secondly purified air may be transferred to the third nanofiber filter layer 140 while simultaneously passing through the third nanofiber filter layer 140. The air A1 with the harmful gas A1-2, odor A1-2, and bad breath A1-2 removed may pass through the third nanofiber filter layer 140 while being simultaneously filtered by the third nanofiber filter layer 140, so that the moisture A1-3 and water vapor A1-3 contained in the air A1 may be removed. The firstly, secondly, and thirdly purified air A1-4 may be transferred to the second support 150 while simultaneously passing through the second support 150 and be discharged to the outside of the second support 150.

According to various embodiments, the nanofiber filter 100 may be manufactured as a single filter by sequentially disposing the first, second and third nanofiber filter layers 120, 130 and 140, and the nanofiber filter 100 may sequentially perform sterilization of the virus A1-1 contained in the air A1, removal of the harmful gas A1-2, removal of the odor A1-2, removal of the bad breath A1-2, removal of the moisture A1-3, and removal of the water vapor A1-3. For example, since the nanofiber filter 100 does not require a plurality of filters which are conventionally required to perform the plurality of functions described above, the manufacturing costs of the product may be reduced.

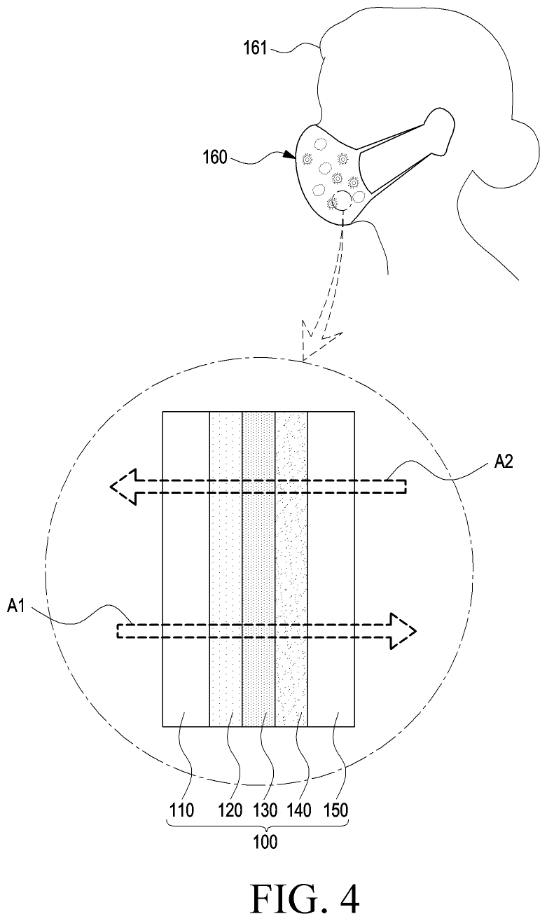

FIG. 4 is a view illustrating a state in which a nanofiber filter 100 is applied to a mask 160 according to various embodiments of the disclosure.

Referring to FIG. 4, according to various embodiments, a nanofiber filter 100 may include first and second supports 110 and 150 and first, second, and third nanofiber filter layers 120, 130, and 140. For example, in the nanofiber filter 100, the first nanofiber filter layer 120, the second nanofiber filter layer 130, and the third nanofiber filter layer 140 may be sequentially disposed toward the rear surface of the first support 110, and the second support 150 may be sequentially disposed on the rear surface of the third nanofiber filter layer 140.

The nanofiber filter 100 having such a structure may be applied to the mask 160. For example, the wearer 161 may wear the mask 160 including the nanofiber filter 100. In this case, the first support 110 may be disposed on the front surface of the nanofiber filter 100 to introduce external air A1. The second support 150 may be disposed on the rear surface of the nanofiber filter 100 while being simultaneously positioned on the nose and mouth of the wearer 161. In this state, when the external air A1 passes through the first support 110 and is introduced into the inside of the nanofiber filter 100, the introduced air A1 may be firstly transmitted through, and simultaneously filtered by, the first nanofiber filter layer 120 formed by electrospinning the photocatalytic nanomaterial mixture. In this case, the first nanofiber filter layer 120 may sterilize the virus (A1-1 of FIG. 1) contained in the air A1.

The firstly purified air A1 may be secondarily transmitted through, and simultaneously filtered by, the second nanofiber filter layer 130 formed by electrospinning the gas suction material mixture. In this case, the second nanofiber filter layer 130 may remove the harmful gas (A1-2 of FIG. 1), odor (A1-2 of FIG. 1) and bad breath (A1-2 of FIG. 1) contained in the air A1.

The firstly and secondly purified air may be thirdly transmitted through, and simultaneously filtered by, the third nanofiber filter layer 140. The third nanofiber filter may be formed by electrospinning the hygroscopic nanomaterial mixture. In this case, the third nanofiber filter layer 140 may remove the moisture (A1-3 of FIG. 1) and water vapor (A1-3 of FIG. 1) contained in the air A1.

The firstly, secondly, and thirdly purified air A1 may pass through the second support 150 and be transferred to the nose and mouth of the wearer 161. In this case, the nose and mouth of the wearer 161 may breathe the firstly, secondly, and thirdly purified air A1. For example, the nose and mouth of the wearer 161 may safely inhale the firstly, secondly, and thirdly purified air A1. In this case, since the air A2 discharged through the nose and mouth of the wearer 161 contains bad breath and moisture, the internal air A2 discharged through the nose and mouth of the wearer 161 may be reversely and sequentially transmitted through, and simultaneously filtered and thus purified by, the third nanofiber filter layer 140, the second nanofiber filter layer 130, and the first nanofiber filter layer 120. The so-purified internal air A2 may pass through the first support 110 while being simultaneously discharged to the outside of the first support 110.

As such, the mask 160 including the nanofiber filter 100 may perform sterilization of the virus (A1-1 of FIG. 1) contained in the external air A1, removal of the harmful gas (A1-2 of FIG. 1), removal of odor (A1-2 of FIG. 1), removal of bad breath (A1-2 of FIG. 1), removal of moisture (A1-3 of FIG. 1), and removal of water vapor (A1-3 of FIG. 1) using the first, second, third nanofiber filter layers 120, 130, and 140 sequentially disposed. Accordingly, when the wearer 161 breathes the external air A1, the mask 160 may block the virus (A1-1 of FIG. 1), harmful gas (A1-2 of FIG. 1), odor (A1-2 of FIG. 1), bad breath (A1-2 of FIG. 1), moisture (A1-3 of FIG. 1), and water vapor (A1-3 of FIG. 1) contained in the air from being inhaled into the body of the wearer 161.

Further, the mask 160 may sterilize the virus contained in the internal air A2 and remove bad breath and moisture discharged through the nose and mouth of the wearer 161 using the first, second, and third nanofiber filter layers 120, 130, and 140 sequentially disposed. Accordingly, the mask 160 may block the virus, bad breath, and moisture contained in the internal air A2 from being discharged to the outside of the mask 160. For example, when a patient with a respiratory disease wears the mask 160, the mask 160 may prevent discharge of various infectious viruses discharged from the wearer's respiratory system, preventing secondary infection to others.

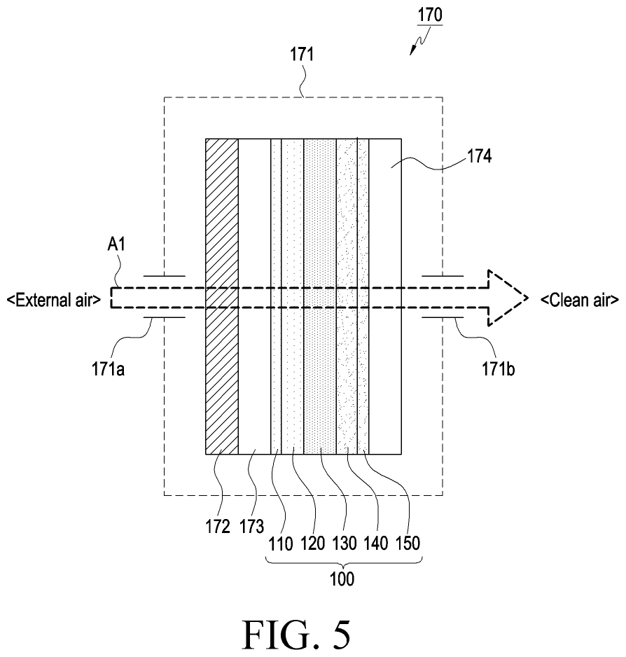

FIG. 5 is a view illustrating a state in which a nanofiber filter 100 is applied to an air purifier 170 according to various embodiments of the disclosure.

Referring to FIG. 5, according to various embodiments, a nanofiber filter 100 may include first and second supports 110 and 150 and first, second, and third nanofiber filter layers 120, 130, and 140. For example, in the nanofiber filter 100, the first nanofiber filter layer 120, the second nanofiber filter layer 130, and the third nanofiber filter layer 140 may be sequentially disposed toward the rear surface of the first support 110, and the second support 150 may be sequentially disposed on the rear surface of the third nanofiber filter layer 140.

The nanofiber filter 100 having such a structure may be applied to the air purifier 170. For example, the air purifier 170 may include a housing 171 including a suction unit 171a and a discharge unit 171b, a pre-filter 172, an LED 173, a nanofiber filter 100, and a fan 174. For example, the pre-filter 172 may be disposed within the housing 171 and may be disposed facing the suction unit 171a of the housing 171. The LED 173 may be disposed on the rear surface of the pre-filter 172 and may be disposed on the front surface of the nanofiber filter 100. The fan 174 may be disposed between the rear surface of the nanofiber filter 100 and the discharge unit 171b.

In this state, the air purifier 170 may be operated by pressing an on/off switch disposed on at least a portion of the housing 171 to switch on. If the on/off switch is turned on, the LED 173 and the fan 174 disposed in the housing 171 may be operated. While the fan 174 is operated, external air A1 may be simultaneously introduced into the inside of the housing 171 through the suction unit 171a of the housing 171.

The introduced air A1 may pass through the first support 110 disposed on the front surface of the nanofiber filter 100 via the pre-filter 172 and the LED 173 and be introduced into the inside of the nanofiber filter 100.

In this case, when the air A1 passes through the first support 110 and is introduced into the inside of the nanofiber filter 100, the introduced air A1 may be firstly transmitted through, and simultaneously filtered by, the first nanofiber filter layer 120. The first nanofiber filter layer 120 may be formed by electrospinning the photocatalytic nanomaterial mixture. In this case, the first nanofiber filter layer 120 may sterilize the virus (A1-1 of FIG. 1) contained in the air A1.

The firstly purified air A1 may be secondarily transmitted through, and simultaneously filtered by, the second nanofiber filter layer 130. The second nanofiber filter layer 130 may be formed by electrospinning the gas suction material mixture. In this case, the second nanofiber filter layer 130 may remove the harmful gas (A1-2 of FIG. 1), odor (A1-2 of FIG. 1) and bad breath (A1-2 of FIG. 1) contained in the air A1.

The firstly and secondly purified air A1 may be thirdly transmitted through, and simultaneously filtered by, the third nanofiber filter layer 140. The third nanofiber filter layer 140 may be formed by electrospinning the hygroscopic nanomaterial mixture. In this case, the third nanofiber filter layer 140 may remove the moisture (A1-3 of FIG. 1) and water vapor (A1-3 of FIG. 1) contained in the air A1.

The firstly, secondly, and thirdly purified air A1 may pass through the second support 150 and be transferred to the fan 174. The fan 174 may discharge the transferred, firstly, secondly, and thirdly purified air (A1-4 of FIG. 1) through the discharge unit 171b of the housing 171 to the outside of the housing 171.

As such, the air purifier 170 including the nanofiber filter 100 may perform sterilization of the virus (A1-1 of FIG. 1) contained in the external air A1, removal of the harmful gas (A1-2 of FIG. 1), removal of odor (A1-2 of FIG. 1), removal of bad breath (A1-2 of FIG. 1), removal of moisture (A1-3 of FIG. 1), and removal of water vapor (A1-3 of FIG. 1) using the first, second, third nanofiber filter layers 120, 130, and 140 sequentially disposed. Therefore, the air purifier 170 may further enhance the function of the air purifier 170 by supplying the clean air (A1-4 of FIG. 1), with the virus (A1-1 of FIG. 1), harmful gas (A1-2 of FIG. 1), odor (A1-2 of FIG. 1), bad breath (A1-2 of FIG. 1), moisture (A1-3 of FIG. 1), and water vapor (A1-3 of FIG. 1) removed.

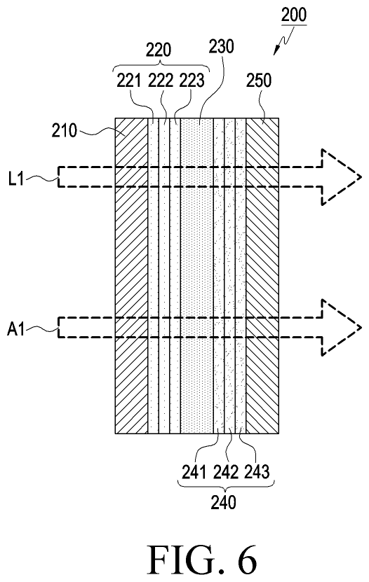

FIG. 6 is a side view illustrating a configuration of a nanofiber filter 200 according to other various embodiments of the disclosure.

Referring to FIG. 6, according to other various embodiments, a nanofiber filter 220 and 240 may include first, second, and third supports 210, 230, and 250 and first and second nanofiber filters 220 and 240. For example, the first, second, and third supports 210, 230, and 250 may include a front surface and a rear surface opposite to the front surface. The first and second nanofiber filters 220 and 240 may also include a front surface and a rear surface opposite to the front surface.

The rear surface of the first support 210 may be disposed facing the front surface of the first nanofiber filter 220, and the front surface of the second support 230 may be disposed facing the rear surface opposite to the front surface of the first nanofiber filter 220. The front surface of the second nanofiber filter 240 may be disposed on the rear surface opposite to the front surface of the second support 230. The front surface of the third support 250 may be disposed on the rear surface opposite to the front surface of the second nanofiber filter 240.

According to various embodiments, the first nanofiber filter 220 may include first, second, and third nanofiber filter layers 221, 222, and 223. For example, the front surface of the first nanofiber filter layer 221 may be disposed facing the rear surface of the first support body 210. The front surface of the second nanofiber filter layer 222 may be disposed facing the rear surface of the first nanofiber filter layer 221. The front surface of the third nanofiber filter layer 223 may be disposed facing the rear surface of the second nanofiber filter layer 240. The front surface of the second support 230 may be disposed facing the rear surface of the third nanofiber filter layer 223.

Further, the second nanofiber filter 240 may include fourth, fifth, and sixth nanofiber filter layers 241, 242, and 243. For example, the front surface of the fourth nanofiber filter layer 241 may be disposed facing the rear surface of the second support body 230. The front surface of the fifth nanofiber filter layer 242 may be disposed facing the rear surface of the fourth nanofiber filter layer 241. The front surface of the sixth nanofiber filter layer 243 may be disposed facing the rear surface of the fifth nanofiber filter layer 242. The front surface of the third support 250 may be disposed facing the rear surface of the sixth nanofiber filter layer 243.

Therefore, the first nanofiber filter 220 including the first, second, and third nanofiber filter layers 221, 222, and 223 may be sequentially disposed between the first and second supports 210 and 230, and the second nanofiber filter 240 including the fourth, fifth, and sixth nanofiber filter layers 241, 242, and 243 may be sequentially disposed between the second and third supports 230 and 250.

According to various embodiments, the first, second, and third supports 210, 230, and 250 and the first and second nanofiber filters 220 and 240 may be attached to each other through an adhesive member or may be attached without the adhesive member.

According to various embodiments, the first, second and third supports 210, 230 and 250 may include at least one of a transparent breathable support and a translucent breathable support. For example, various breathable supports transmitting air A1 or light C1 may be applied as the first, second, and third supports 210, 230, and 250. In this embodiment, an example in which the first, second, and third supports 210, 230 and 250 are breathable transparent supports is described.

According to various embodiments, the first, second, and third supports 210, 230 and 250 may include at least one of a hydrophilic polymer material, a hydrophobic polymer material, or an easily decomposable polymer material. For example, the hydrophilic polymer material may include polyacrylonitrile (PAN). The hydrophilic polymer material may be other various hydrophilic polymer materials than polyacrylonitrile (PAN).

Further, the hydrophobic polymer material may include at least one of polyvinylylene fluoride (PVDF) or polyethylene terephthalate (PET). Likewise, the hydrophobic polymer material may be other various hydrophobic polymer materials than the above-disclosed material.

Further, the easily decomposable polymer material may include polyvinyl alcohol (PVA). Similarly, the easily decomposable polymer material may be other various readily decomposable polymer materials than polyvinyl alcohol (PVA).

According to various embodiments, the first, second, and third supports 210, 230 and 250 may include at least one of a metal mesh member or a breathable transparent film to transmit air A1 and light C1. In this embodiment, an example in which the first, second, and third supports 210, 230 and 250 are breathable transparent films is described.

According to various embodiments, the first support 210 may transmit air A1 containing virus, harmful gas, odor, bad breath, moisture and water vapor and transfer it to the first nanofiber filter 220. The air A1 passing through the first nanofiber filter 220 may pass through the second support 230 and be transferred to the second nanofiber filter 240. As such, the air A1 passing through the first and second nanofiber filters 220 and 240 and the second support 230 may pass through the third support 250 and be discharged to the outside of the third support 250.

In this case, the first nanofiber filter layer 221 of the first nanofiber filter 220 may be formed by electrospinning a photocatalytic nanomaterial mixture. The air A1 passing through the first support 210 may be transmitted, and simultaneously filtered firstly by, the first nanofiber filter layer 221, sterilizing the virus (A1-1 of FIG. 1) contained in the air A1.

The firstly purified air may pass through the second nanofiber filter layer 222 of the first nanofiber filter 220. The second nanofiber filter layer 222 may be formed by electrospinning a gas suction material mixture. While the firstly purified air passes through the second nanofiber filter layer 222, the harmful gas (A1-2 of FIG. 1), odor (A1-2 of FIG. 1), and bad breath (A1-2 of FIG. 1) contained in the air may be simultaneously filtered secondly and removed by the second nanofiber filter layer 222.

The firstly and secondly purified air (A1-4 in FIG. 1) may pass through the third nanofiber filter layer 223 of the first nanofiber filter 220. For example, the third nanofiber filter layer 223 may be formed by electrospinning a hygroscopic nanomaterial mixture. While the firstly and secondly purified air passes through the third nanofiber filter layer 223, the moisture (A1-3 of FIG. 1) and water vapor (A1-3 of FIG. 1) contained in the air A1 may be simultaneously filtered thirdly and removed by the third nanofiber filter layer 223.

The firstly, secondly, and thirdly purified air may pass through the second support 230 while being simultaneously transferred thereto, and may be transferred to the second nanofiber filter 240.

In this case, the fourth nanofiber filter layer 241 of the second nanofiber filter 240 may be formed by electrospinning a photocatalytic nanomaterial mixture. The air A1 passing through the second support 230 may be transmitted, and simultaneously filtered fourthly by, the fourth nanofiber filter layer 241, sterilizing the virus (A1-1 of FIG. 1) contained in the air A1.

The fourthly purified air A1 may pass through the fifth nanofiber filter layer 242 of the second nanofiber filter 240. The fifth nanofiber filter layer 242 may be formed by electrospinning a gas suction material mixture. While the fourthly purified air passes through the fifth nanofiber filter layer 242, the harmful gas (A1-2 of FIG. 1), odor (A1-2 of FIG. 1), and bad breath (A1-2 of FIG. 1) contained in the air may be simultaneously filtered fifthly and removed by the fifth nanofiber filter layer 242.

The fourthly and fifthly purified air A1 may pass through the sixth nanofiber filter layer 243 of the second nanofiber filter 240. For example, the sixth nanofiber filter layer 243 may be formed by electrospinning a hygroscopic nanomaterial mixture. While the fourthly and fifthly purified air passes through the sixth nanofiber filter layer 243, the moisture (A1-3 of FIG. 1) and water vapor (A1-3 of FIG. 1) contained in the air A1 may be simultaneously filtered sixthly and removed by the sixth nanofiber filter layer 243.

The firstly, secondly, thirdly, fourthly, fifthly, and sixthly purified air (A1-4 of FIG. 1) may pass through the third support 250 while being simultaneously transferred thereto, and may be discharged to the outside of the third support 250.

The air (A1-4 of FIG. 1) passing through the third support 250 may be discharged to the outside, with the virus sterilized and the harmful gas, odor, bad breath, moisture, and water vapor removed by the first, second, third, fourth, fifth, and sixth nanofiber filter layers 221, 222, 223, 241, 242, and 243.

According to various embodiments, the first, second, third, fourth, fifth, and sixth nanofiber filter layers 221, 222, 223, 241, 242, and 243 may be manufactured by an electrospinning device. The electrospinning device may be at least partially identical or similar in configuration to that of the above-described electrospinning device. Therefore, since the configuration of the electrospinning device may easily be understood from the above-described embodiment of the electrospinning device, no detailed description thereof is given below.

As such, the nanofiber filter 220 and 240 may be configured as a single filter by sequentially disposing the first nanofiber filter 220 including the first, second, and third nanofiber filter layers 221, 222, and 223 and the second nanofiber filter 240 including the fourth, fifth, and sixth nanofiber filter layers 241, 242, and 243, so that the nanofiber filter 200 may repeatedly perform sterilization of the virus (A1-1 of FIG. 1) contained in the air A1, removal of the harmful gas (A1-2 of FIG. 1), removal of odor (A1-2 of FIG. 1), removal of bad breath (A1-2 of FIG. 1), removal of moisture (A1-3 of FIG. 1), and removal of water vapor (A1-3 of FIG. 1). Therefore, the nanofiber filter 200 may further enhance the function of purifying the air.

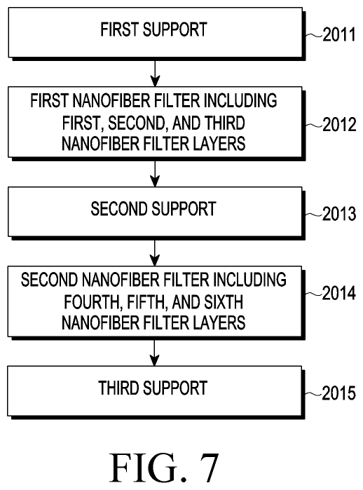

FIG. 7 is a flowchart illustrating a method for manufacturing a nanofiber filter 200 according to other various embodiments of the disclosure.

Referring to FIG. 7, according to other various embodiments, the first support 210 may be prepared to manufacture the nanofiber filter 200 (2011). For example, the first support 210 may include a front surface and a rear surface opposite to the front surface.

The first nanofiber filter 220 including the first, second, and third nanofiber filter layers 221, 222, and 223 may be disposed on the rear surface of the first support 210 (2012). For example, the rear surface of the first support 210 and the front surface of the first nanofiber filter 220 may be disposed to face each other. The first nanofiber filter 220 includes a front surface and a rear surface. The front surface of the first nanofiber filter 220 is disposed on the rear surface of the first support 210.

The second support 230 may be disposed on the rear surface opposite to the front surface of the first nanofiber filter 220. For example, the rear surface of the first nanofiber filter layer 221 and the front surface of the second support 230 may be disposed to face each other (2013).

According to various embodiments, the first nanofiber filter layer 221 formed by electrospinning the photocatalytic nanomaterial mixture may be disposed on the rear surface of the first support 210. For example, the rear surface of the first support 210 and the front surface of the first nanofiber filter layer 221 may be disposed to face each other.

The second nanofiber filter layer 222 formed by electrospinning the gas suction material mixture may be disposed on the rear surface opposite to the front surface of the first nanofiber filter layer 221. For example, the rear surface of the first nanofiber filter layer 222 and the front surface of the second nanofiber filter layer 222 may be disposed to face each other.

The third nanofiber filter layer 223 formed by electrospinning the hygroscopic nanomaterial mixture may be disposed on the rear surface opposite to the front surface of the second nanofiber filter layer 222. For example, the rear surface of the second nanofiber filter layer 222 and the front surface of the third nanofiber filter layer 223 may be disposed to face each other.

The second support 230 may be disposed on the rear surface opposite to the front surface of the third nanofiber filter layer 223. For example, the rear surface of the third nanofiber filter layer 223 and the front surface of the second support 230 may be disposed to face each other.

The front surface of the second nanofiber filter 240 including the fourth, fifth, and sixth nanofiber filter layers 241, 242, and 243 may be disposed on the rear surface opposite to the front surface of the second support 230 (2014). The front surface of the third support 250 may be disposed to face the rear surface opposite to the front surface of the second nanofiber filter 240.

According to various embodiments, the second support 230 may be disposed on the rear surface opposite to the front surface of the first nanofiber filter 220. For example, the rear surface of the first nanofiber filter 220 and the front surface of the second support 230 may be disposed to face each other.

According to various embodiments, the fourth nanofiber filter layer 241 formed by electrospinning the photocatalytic nanomaterial mixture may be disposed on the rear surface of the second support 230. For example, the rear surface of the second support 230 and the front surface of the fourth nanofiber filter layer 241 may be disposed to face each other.

The fifth nanofiber filter layer 242 formed by electrospinning the gas suction material mixture may be disposed on the rear surface opposite to the front surface of the fourth nanofiber filter layer 241. For example, the rear surface of the fourth nanofiber filter layer 241 and the front surface of the fifth nanofiber filter layer 242 may be disposed to face each other.

The sixth nanofiber filter layer 243 formed by electrospinning the hygroscopic nanomaterial mixture may be disposed on the rear surface opposite to the front surface of the fifth nanofiber filter layer 242. For example, the rear surface of the fifth nanofiber filter layer 242 and the front surface of the sixth nanofiber filter layer 243 may be disposed to face each other.

The third support 250 may be disposed on the rear surface opposite to the front surface of the sixth nanofiber filter layer 243. For example, the rear surface of the sixth nanofiber filter layer 243 and the front surface of the third support 250 may be disposed to face each other.

For example, the first support 210 may be disposed on the front surface of the first nanofiber filter 220, the second support 230 may be disposed on the rear surface of the first nanofiber filter 220 and 240, the front surface of the second nanofiber filter 240 may be disposed on the rear surface of the second support 230, and the third support 250 may be disposed on the rear surface of the second nanofiber filter 240 (2015).

In the nanofiber filter 200, when air is introduced through the first support 210, the first support 210 may transmit and simultaneously transfer the air to the first nanofiber filter 220. The air may sequentially pass through the first, second, and third nanofiber filter layers 221, 222, and 223 included in the first nanofiber filter 220 and 240. For example, the air may be firstly filtered by the first nanofiber filter layer 221, sterilizing the virus (A1-1 of FIG. 1) contained in the air A1. Further, the air A1 may be secondly filtered by the second nanofiber filter layer 222, removing the harmful gas (A1-2 of FIG. 1), odor (A1-2 of FIG. 1) and bad breath (A1-2 of FIG. 1) contained in the air A1. Further, the air A1 may be thirdly filtered by the third nanofiber filter layer 223, removing the moisture (A1-3 of FIG. 1) and water vapor (A1-3 of FIG. 1) contained in the air A1. The firstly, secondly, and thirdly purified air (A1-4 of FIG. 1) may be transferred to the second support 230 while simultaneously passing through the second support 230 and be transferred to the second nanofiber filter 240. In this case, the transferred air may sequentially pass through the fourth, fifth, and sixth nanofiber filter layers 241, 242, and 243 included in the second nanofiber filter 240. The air may be fourthly filtered by the fourth nanofiber filter layer 241, once more sterilizing the virus (A1-1 of FIG. 4) contained in the air A1. Further, the air may be fifthly filtered by the fifth nanofiber filter layer 242, once more removing the harmful gas (A1-2 of FIG. 1), odor (A1-2 of FIG. 1) and bad breath (A1-2 of FIG. 1) contained in the air. Further, the air A1 may be thirdly filtered by the sixth nanofiber filter layer 243, once more removing the moisture (A1-3 of FIG. 1) and water vapor (A1-3 of FIG. 1) contained in the air. The fourthly, fifthly, and sixthly purified air (A1-4 of FIG. 1) may be transferred to the third support 250 while simultaneously passing through the third support 250 and be discharged to the outside of the third support 250.

According to various embodiments, the nanofiber filter 200 may be manufactured as a single filter by sequentially disposing the first, second, third, fourth, fifth, and sixth nanofiber filter layers 221, 222, 223, 241, 242, and 243, so that the nanofiber filter 200 may sequentially and repeatedly perform sterilization of the virus (A1-1 of FIG. 1) contained in the air A1, removal of the harmful gas (A1-2 of FIG. 1), removal of odor (A1-2 of FIG. 1), removal of bad breath (A1-2 of FIG. 1), removal of moisture (A1-3 of FIG. 1), and removal of water vapor (A1-3 of FIG. 1). Therefore, the nanofiber filter 200 may further enhance the function of purifying the air.

According to various embodiments, the nanofiber filter 200 may be applied to at least one of a mask or an air purifier. The mask or the air purifier may be identical or similar in at least one of the components to the mask 160 or air purifier 170 of FIGS. 4 and 5, and no duplicate description thereof is given below.

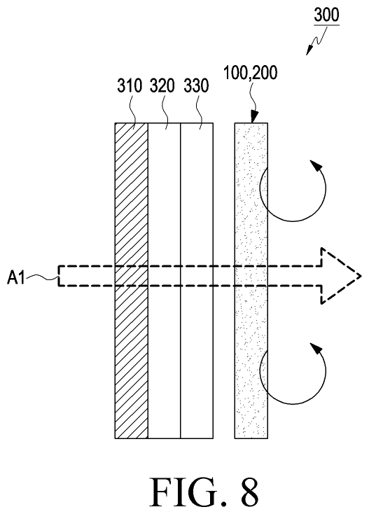

FIG. 8 is a view illustrating a regeneration device 300 of a nanofiber filter 100 or 200 according to various embodiments of the disclosure.

Referring to FIG. 8, according to various embodiments, a regeneration device 300 of a nanofiber filter 100 or 200 may include an air permeable membrane 310, a plurality of fans 320, and an LED 330. For example, the air permeable membrane 310 may include a front surface and a rear surface opposite to the front surface and transmit external air A1. The plurality of fans 320 may be disposed on the rear surface of the air permeable membrane 310. The plurality of fans 320 may be activated according to ON or OFF of an on/off switch. The LED 330 may be disposed on the rear surface of the plurality of fans 320. The LED 330 may generate OH radicals according to ON or OFF of the on/off switch. In this state, a mask (e.g., the mask 160 of FIG. 4) including the nanofiber filter 100 or 200 may be attached to and detached from the rear surface of the plurality of fans 320. For example, the front surface of the mask (e.g., the mask 160 of FIG. 4) may be attached to or detached from the rear surface of the plurality of fans 320.

According to various embodiments, after use of the mask (e.g., the mask 160 of FIG. 4), the wearer (e.g., the wearer 161 of FIG. 4) may mount the mask (e.g., the mask 160 of FIG. 4) on the regeneration device 300 of the nanofiber filter 100 or 200 to clean the nanofiber filter 100 or 200 included in the mask (e.g., the mask 160 of FIG. 4). For example, the front surface of the nanofiber filter 100 or 200 included in the mask (e.g., the mask 160 of FIG. 4) may be disposed on the rear surface of the plurality of fans 320 while the mask (e.g., the mask 160 of FIG. 4) is simultaneously mounted on the regeneration device 300 of the nanofiber filter 100 or 200.

If the on/off switch is turned on in this state, the plurality of fans 320 may be activated and may simultaneously introduce the external air A1 through the air permeable membrane 310 to the inside of the regeneration device 300. In this case, the air permeable membrane 310 may remove the dust and foreign bodies contained in the external air A1. Thus, it is possible to prevent contamination of the plurality of fans 320 and the nanofiber filter 100 or 200.

Simultaneously, the LED 330 may emit light, and the nanofiber filter 100 or 200 containing photocatalyst may be activated by the light energy of the LED, generating OH radicals. For example, the OH radicals generated by the nanofiber filter 100 or 200 have a very short lifespan (e.g., micro-seconds) and thus would not move along the introduced air A1 or be exposed to the human body, but may decompose the viruses (or organic substances, such as harmful gases) filtered or suctioned onto the surface near the nanofiber filter 100 or 200 into carbon dioxide that is harmless to the human body. For example, the OH radicals, along with the air A1, may pass through the nanofiber filter 100 or 200 while simultaneously sterilizing the viruses included in the nanofiber filter 100 or 200. The nanofiber filter 100 or 200 may be reused after sterilization of viruses by the OH radicals. Therefore, the mask (e.g., the mask 160 of FIG. 4) including the nanofiber filter 100 or 200 in which the virus is sterilized may be separated from the regeneration device 300 and reused.

As such, the regeneration device 300 of the nanofiber filter 100 or 200 may sterilize the virus included in the nanofiber filter 100 or 200 of the used mask (e.g., the mask 160 of FIG. 4), allowing for reuse of the mask (e.g., the mask 160 of FIG. 4) which is supposed to be discarded. Thus, it is possible to relieve the wearer (e.g., the wearer 161 of FIG. 4) of the cost burden for purchasing a new mask and reduce environmental contamination due to waste of the mask (e.g., the mask 160 of FIG. 4).

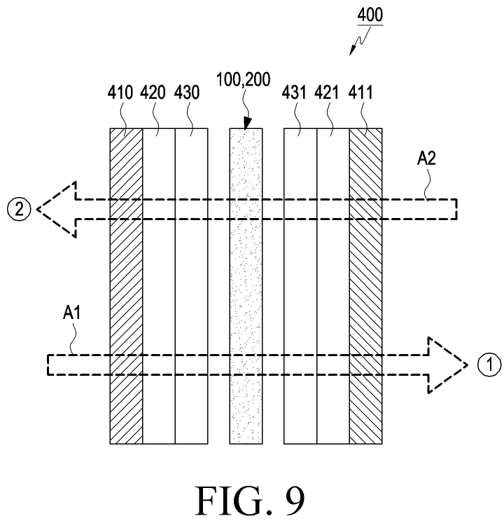

FIG. 9 is a view illustrating another embodiment of a regeneration device 400 of a nanofiber filter 100 or 200 according to various embodiments of the disclosure.

Referring to FIG. 9, according to other various embodiments, a regeneration device 400 of a nanofiber filter 100 or 200 may include first and second air permeable membranes 410 and 411, first and second fans 420 and 421, and first and second LEDs 430 and 431. For example, the first and second air permeable membranes 410 and 411 may include a front surface and a rear surface opposite to the front surface and transmit external air A1.

The first fan 420 may be disposed on the rear surface of the first air permeable membrane 410, and the first LED 430 may be disposed on the rear surface of the first fan 420. The second LED 431 may be disposed on the rear surface of the first LED 430, and the second fan 421 may be disposed on the rear surface of the second LED 431. The second air permeable membrane 411 may be disposed on the rear surface of the second fan 421. A mask (e.g., the mask 160 of FIG. 4) including the nanofiber filter 100 or 200 may be attached or detached between the first and second LEDs 430 and 431.

According to various embodiments, the first and second fans 420 and 421 may be operated according to ON or OFF of an on/off switch. The first and second LEDs 430 and 431 may generate OH radicals according to ON or OFF of the on/off switch.

According to various embodiments, after use of the mask (e.g., the mask 160 of FIG. 4), the wearer (e.g., the wearer 161 of FIG. 4) may mount the used mask (e.g., the mask 160 of FIG. 4) between the first and second LEDs 430 and 431 to clean the nanofiber filter 100 or 200 included in the mask (e.g., the mask 160 of FIG. 4). For example, while the mask (e.g., the mask 160 of FIG. 4) is simultaneously mounted between the first and second LEDs 430 and 431, the front surface of the nanofiber filter 100 or 200 included in the mask (e.g., the mask 160 of FIG. 4) may be disposed facing the rear surface of the first LED 430, and the rear surface of the nanofiber filter 100 or 200 may be disposed facing the front surface of the second LED 431.

If the on/off switch is turned on in this state, the first and second fans 420 and 421 may be operated while the first fans 420 and 421 may simultaneously introduce the external air A1 to the first air permeable membrane 410 or 411 in a first direction \textcircled{1}. For example, as the first fan 420 operates, the external air A1 may pass through the first air permeable membrane 410 or 411 and be introduced into the inside of the regeneration device 400. Simultaneously, the second fan 421 may introduce the external air A2 to the second air permeable membrane 410 or 411 in a second direction \textcircled{2} opposite to the first direction \textcircled{1}. For example, as the second fan operates, the external air A2 may pass through the second air permeable membrane 410 or 411 and be introduced into the inside of the regeneration device 400.

In this case, the first and second air permeable membranes 410 and 411 may remove the dust and foreign bodies contained in the external air A1 and A2. Thus, it is possible to prevent contamination of the first and second fans 420 and 421 and the nanofiber filter 100 or 200.

Simultaneously, the first and second LEDs 430 and 431 may emit light, and the nanofiber filter 100 or 200 containing photocatalyst may be activated by the light energy of the LEDs, generating first and second OH radicals to sterilize virus. For example, the first and second OH radicals generated by the nanofiber filter 100 or 200, along with the air A1 and A2 introduced in the first and second directions \textcircled{1} and \textcircled{2}, may be transferred to the nanofiber filter 100 or 200 and sterilize the virus filtered or suctioned onto the surface near the nanofiber filter 100 or 200. For example, the first and second OH radicals, along with the introduced air A1 and A2, may pass through the nanofiber filter 100 or 200 while simultaneously sterilizing the virus included in the nanofiber filter 100 or 200. Thus, the nanofiber filter 100 or 200 may be reused after sterilization of viruses by the first and second OH radicals.

As such, the regeneration device 400 of the nanofiber filter 100 or 200 may sterilize the virus included in the nanofiber filter 100 or 200 of the used mask 160, so that the regeneration device 400 may further enhance virus sterilization of the nanofiber filter 100 or 200, thus further enhancing reuse of the mask 160.

According to various embodiments of the disclosure, a nanofiber filter (e.g., the nanofiber filter 100 of FIG. 1) may include a first support (e.g., the first support 110 of FIG. 1) including a front surface and a rear surface opposite to the front surface, a first nanofiber filter layer (e.g., the first nanofiber filter layer 120 of FIG. 1) disposed on the rear surface of the first support, a second nanofiber filter layer (e.g., the second nanofiber filter layer 130 of FIG. 1) disposed on a rear surface of the first nanofiber filter layer, a third nanofiber filter layer (e.g., the third nanofiber filter layer 140) disposed on a rear surface of the second nanofiber filter layer, and a second support (e.g., the second support 150 of FIG. 1) disposed on a rear surface of the third nanofiber filter layer.

According to various embodiments of the disclosure, each the first support and the second support may comprise at least one of a transparent breathable support or a translucent breathable support.

According to various embodiments of the disclosure, each the first support and the second support may comprise at least one of a hydrophilic polymer material, a hydrophobic polymer material, or a decomposable polymer material.

According to various embodiments of the disclosure, each the first support and the second support may comprise at least one of a metal mesh member or a breathable transparent film.

According to various embodiments of the disclosure, the first nanofiber filter layer may comprise an electrospun a photocatalytic nanomaterial mixture.

According to various embodiments of the disclosure, the photocatalytic nanomaterial mixture may comprise at least one of UV-sensitive photocatalytic nanoparticles or visible light-sensitive photocatalytic nanoparticles.

According to various embodiments of the disclosure, the second nanofiber filter layer may comprise an electrospun gas suction material mixture.

According to various embodiments of the disclosure, the third nanofiber filter layer may comprise an electrospun hygroscopic nanomaterial mixture.

According to various embodiments of the disclosure, the nanofiber filter may be configured to filter at least one of a virus, gas, odor, moisture, and water vapor from air.

According to various embodiments of the disclosure, a method for manufacturing a nanofiber filter may include preparing a first support including a front surface and a rear surface opposite to the front surface, disposing a first nanofiber filter layer formed by electrospinning a photocatalytic nanomaterial mixture on the rear surface of the first support, disposing a second nanofiber filter layer formed by electrospinning a gas suction material mixture on a rear surface of the first nanofiber filter layer, disposing a third nanofiber filter layer formed by electrospinning a hygroscopic nanomaterial mixture on a rear surface of the second nanofiber filter layer, and disposing a second support on a rear surface of the third nanofiber filter layer.

According to various embodiments of the disclosure, a nanofiber filter (e.g., the nanofiber filter 200 of FIG. 6) may include a first support (e.g., the first support 210 of FIG. 6) including a front surface and a rear surface opposite to the front surface, a first nanofiber filter (e.g., the first nanofiber filter 220 of FIG. 6) including first, second, and third nanofiber filter layers disposed on the rear surface of the first support, a second support (e.g., the second support 230 of FIG. 6) disposed on a rear surface of the first nanofiber filter, a second nanofiber filter (e.g., the second nanofiber filter 240 of FIG. 6) including fourth, fifth, and sixth nanofiber filter layers disposed on a rear surface of the second support, and a third support (e.g., the third support 250 of FIG. 6) disposed on a rear surface of the second nanofiber filter.

According to various embodiments of the disclosure, the first and fourth nanofiber filter layers may be formed by electrospinning a photocatalytic nanomaterial mixture.

According to various embodiments of the disclosure, the second and fifth nanofiber filter layers may be a nanofiber filter formed by electrospinning a gas suction material mixture.

According to various embodiments of the disclosure, the third and sixth nanofiber filter layers may be formed by electrospinning a hygroscopic nanomaterial mixture.

According to various embodiments of the disclosure, a nanofiber filter may include a first support including a front surface and a rear surface opposite to the front surface, a first nanofiber filter layer disposed on the rear surface of the first support, a second nanofiber filter layer disposed on a rear surface of the first nanofiber filter layer, a third nanofiber filter layer disposed on a rear surface of the second nanofiber filter layer, and a second support disposed on a rear surface of the third nanofiber filter layer. At least one of the first nanofiber filter layer, the second nanofiber filter layer, and the third nanofiber filter layer may be formed by electrospinning a photocatalytic nanomaterial mixture.

According to various embodiments of the disclosure, a method for manufacturing a nanofiber filter may include preparing a first support including a front surface and a rear surface opposite to the front surface, disposing a first nanofiber filter including a first nanofiber filter layer formed by electrospinning a photocatalytic nanomaterial mixture on the rear surface of the first support, a second nanofiber filter layer formed by electrospinning a gas suction material mixture, and a third nanofiber filter layer formed by electrospinning a hygroscopic nanomaterial mixture, disposing a second support on a rear surface of the first nanofiber filter, disposing a second nanofiber filter including a fourth nanofiber filter layer formed by electrospinning a photocatalytic nanomaterial mixture on a rear surface of the second support, a fifth nanofiber filter layer formed by electrospinning a gas suction material mixture, and a sixth nanofiber filter layer formed by electrospinning a hygroscopic nanomaterial mixture, and disposing a third support on a rear surface of the second nanofiber filter.

It is apparent to one of ordinary skill in the art that the nanofiber filter and method for manufacturing the same according to various embodiments of the disclosure as described above are not limited to the above-described embodiments and those shown in the drawings, and various changes, modifications, or alterations may be made thereto without departing from the scope of the disclosure.

What is claimed is:

1. A nanofiber filter comprising:
a first support having a front surface and a rear surface opposite to the front surface;
a first nanofiber filter layer disposed on the rear surface of the first support;
a second nanofiber filter layer disposed on a rear surface of the first nanofiber filter layer;
a third nanofiber filter layer disposed on a rear surface of the second nanofiber filter layer; and
a second support disposed on a rear surface of the third nanofiber filter layer.

2. The nanofiber filter of claim 1, wherein each the first support and the second support comprises at least one of a transparent breathable support or a translucent breathable support.

3. The nanofiber filter of claim 1, wherein each of the first support and the second support comprises at least one of a hydrophilic polymer material, a hydrophobic polymer material, or a decomposable polymer material.

4. The nanofiber filter of claim 1, wherein each of the first support and the second support comprises at least one of a metal mesh member or a breathable transparent film.

5. The nanofiber filter of claim 1, wherein the first nanofiber filter layer comprises an electrospun photocatalytic nanomaterial mixture.

6. The nanofiber filter of claim 5, wherein the photocatalytic nanomaterial mixture comprises at least one of UV-sensitive photocatalytic nanoparticles or visible light-sensitive photocatalytic nanoparticles.

7. The nanofiber filter of claim 1, wherein the second nanofiber filter layer comprises an electrospun gas suction material mixture.

8. The nanofiber filter of claim 1, wherein the third nanofiber filter layer comprises an electrospun hygroscopic nanomaterial mixture.

9. The nanofiber filter of claim 1, wherein the nanofiber filter is configured to filter at least one of a virus, gas, odor, moisture, and water vapor from air.

10. A method for manufacturing a nanofiber filter, the method comprising:
disposing a first nanofiber filter layer on a rear surface of a first support formed by electrospinning a photocatalytic nanomaterial mixture;
disposing a second nanofiber filter layer on a rear surface of the first nanofiber filter layer formed by electrospinning a gas suction material mixture;
disposing a third nanofiber filter layer on a rear surface of the second nanofiber filter layer formed by electrospinning a hygroscopic nanomaterial mixture; and
disposing a second support on a rear surface of the third nanofiber filter layer.

11. The method of claim 10, wherein each of the first support and the second support comprises at least one of a transparent breathable support or a translucent breathable support.

12. The method of claim 10, wherein each of the first support and the second support comprises at least one of a hydrophilic polymer material, a hydrophobic polymer material, or an easily decomposable polymer material.

13. The method of claim 10, wherein each of the first support and the second support comprises at least one of a metal mesh member or a breathable transparent film.

14. The method of claim 13, wherein the photocatalytic nanomaterial mixture comprises at least one of UV-sensitive photocatalytic nanoparticles or visible light-sensitive photocatalytic nanoparticles.

15. The method of claim 10, wherein the nanofiber filter is configured to filter at least one of a virus, gas, odor, moisture, and water vapor from air.

16. A nanofiber filter comprising:
a first support having a front surface and a rear surface opposite to the front surface;
a first nanofiber filter including first, second, and third nanofiber filter layers disposed on the rear surface of the first support;
a second support disposed on a rear surface of the first nanofiber filter;
a second nanofiber filter including fourth, fifth, and sixth nanofiber filter layers disposed on a rear surface of the second support; and
a third support disposed on a rear surface of the second nanofiber filter.

17. The nanofiber filter of claim 16, wherein the first and fourth nanofiber filter layers comprises an electrospun photocatalytic nanomaterial mixture.

18. The nanofiber filter of claim 16, wherein the second and fifth nanofiber filter layers comprises an electrospun gas suction material mixture.

19. The nanofiber filter of claim 16, wherein the third and sixth nanofiber filter layers comprises an electrospun hygroscopic nanomaterial mixture.

20. A method for manufacturing a nanofiber filter, the method comprising:
disposing a first nanofiber filter including a first nanofiber filter layer formed by electrospinning a photocatalytic nanomaterial mixture on the rear surface of the first support, a second nanofiber filter layer formed by electrospinning a gas suction material mixture, and a third nanofiber filter layer formed by electrospinning a hygroscopic nanomaterial mixture;
disposing a second support on a rear surface of the first nanofiber filter;
disposing a second nanofiber filter including a fourth nanofiber filter layer formed by electrospinning a photocatalytic nanomaterial mixture on a rear surface of the second support, a fifth nanofiber filter layer formed by electrospinning a gas suction material mixture, and a sixth nanofiber filter layer formed by electrospinning a hygroscopic nanomaterial mixture; and
disposing a third support on a rear surface of the second nanofiber filter.
